(19) United States
(12) Patent Application Publication
(10) Pub. No.: US 2005/0118209 A1
(43) Pub. Date: Jun. 2, 2005

(54) Cosmetic or dermatological preparations for preventing damages to skin caused by peroxides

(75) Inventors: Axel Jentszch, Mannheim (DE); Sylke Haremza, Neckargemund (DE); Gerhard Wagenblast, Wachenheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 10/500,459

(22) PCT Filed: Jan. 3, 2003
(86) PCT No.: PCT/EP03/00014

(30) Foreign Application Priority Data
Jan. 18, 2002 (DE) 102 02 065.5
Jan. 28, 2002 (DE) 102 03 414.1

(51) Int. Cl. A61K 31/69; A61K 7/00

(52) U.S. Cl. 424/401; 514/64

(57) ABSTRACT

The invention relates to cosmetic or dermatological preparations that arc characterized by having a content of: a) at least one antioxidant that acts as an 0- or C-radical scavenger, and; b) at least one organic, boron-containing compound that reduces the peroxides or hydroperoxides to the corresponding alcohols without forming active radical subsequent stages.

The invention relates to the use of peroxide decomposers and of a combination of antioxidants and peroxide decomposers which react with peroxides or hydroperoxides, by reduction without the formation of free radical consecutive stages with the peroxides, more rapidly than compounds containing sulfur intrinsic to the skin, and to cosmetic and dermatological preparations which comprise these peroxide decomposers.

The human skin is subject to certain aging processes, some of which are to be attributed to intrinsic processes (chronoaging) and some of which are to be attributed to exogenous factors (environmental, e.g. photoaging). In addition, temporary and also permanent changes in the appearance of the skin can arise, such as acne, greasy or dry skin, keratoses, rosaceae, light-sensitive, inflammatory, erythematous, allergic or autoimmune reactions, such as dermatoses, photodermatoses and others, the exact causes of which and factors which influence them often only being partly understood.

Exogenous factors include, in particular, sunlight or artificial radiation sources with a comparable spectrum, and compounds which can arise as a result of the radiation, such as undefined reactive photoproducts, which may also be free radical or ionic. However, these factors also include harmful or reactive compounds such as ozone, free radicals, for example the hydroxyl radical, singlet oxygen and other reactive oxygen or nitrogen compounds, cigarette smoke, natural and synthetic toxins, and others which interfere with the natural physiology or morphology of the skin. The effect of these factors may result inter alia in direct damage to the DNA of the skin cells, and to the collagen, elastin or glycosaminoglycan molecules of the extracellular matrix which are responsible for the firmness of the skin. Moreover, signal transduction chains may be affected, resulting in the activation of harmful factors, e.g. matrix-degrading enzymes. Important representatives of these enzymes are the matrix metalloproteinases (MMPs, e.g. collagenases, gelatinases, stromelysines), the activity of which is additionally regulated by TIMPs (tissue inhibitor of matrix metalloproteinases).

In addition, the harmful effects lead to damage of the cells of the skin itself. As a consequence thereof, the regeneration ability of the skin, for example, is reduced.

A further consequence may be inflammatory reactions, and, inter alia, immunoregulatory compounds, such as interleukins, prostaglandins and histamines, are released. As a result, immunocompetent cells are attracted, inter alia, and the inflammatory reaction is intensified.

The consequences of aging are thinning of the skin, weaker meshing of epidermis and dermis, reduction in cell number and in supplying blood vessels. The aging processes lead to the formation of fine lines and wrinkles, the skin becomes leathery, yellowish and starts to sag, and pigment disorders arise.

Compounds which have an antioxidative effect are often used in dermatological or cosmetic preparations for protecting against decay. Moreover, they can, however, also be used in order to reduce harmful or undesired oxidative processes which occur in human or animal skin. It is known that such processes play a significant role in skin aging. The skin is exposed to permanent oxidative stress by the formation of peroxides and hydroperoxides, some of which originate from the external environment of the skin, but some of which are also formed endogenously. In order to counteract this stress, the skin has a large number of its own protective mechanisms. These protective mechanisms, however, are insufficient to prevent oxidative processes in the skin completely. By contrast, it is generally assumed that these very oxidative processes make a significant contribution to skin aging, but also to general or pathological changes in the skin.

In particular, the importance of lipid peroxidation for aging is generally recognized. The toxic effect of lipid hydroperoxides and their decomposition products has inter alia been described by W. A. Prior (ACS Sysup. Ser. (1985), 277, 77-96). For the decomposition of peroxides, hydroperoxides or hydrogen peroxide, various systems have also been described in connection with cosmetics, for example the use of metallophosphyrines (JP 3273082), phytic acid zinc salts (JP 08104635), catalase (JP 08175035) and other enzymes (JP 67165553). In addition, JP 06345797 discloses the use of cysteine-containing dipeptides for the bleaching of skin, for the prevention of lipid peroxidation and for the decomposition of lipid peroxides. To aid the endogenous protective mechanisms, constituents with an antioxidative effect, i.e. effective as O- or C-free radical scavengers, are therefore added to cosmetic and dermatological preparations (e.g. DE 19739349). However, the effect actually achieved has hitherto fallen short of that hoped for. In particular, an increase in the added amount of antioxidant does not usually achieve a correspondingly higher antioxidative effect.

It is an object of the present invention to provide active ingredients for cosmetic or dermatological preparations with which the antioxidative effect can be considerably increased.

It is also an object to provide active ingredients for cosmetic or dermatological preparations which protect the skin against oxidative damage.

In general, the mechanism of the formation of peroxide or hydroperoxide conforms to the following scheme

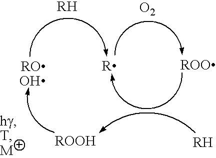

While the customary antioxidants are essentially O- or C-free radical scavengers, it is an object of the invention to prevent skin damage more efficiently by further measures by intervention in the mechanism of this scheme additionally at another site. For this, an ionic and reducing attack according to the following scheme was suitable.

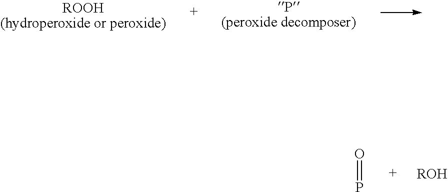

It has now been found that the use of a reducing peroxide decomposer has an excellent effect. In addition, it has been found that the use of a combination of an antioxidant as free radical scavenger and a reducing peroxide decomposer has an excellent synergistic effect. In this case, the peroxide decomposer must be chosen so that it is significantly more reactive in vitro than correspondingly effective sulfur-containing compounds intrinsic to the skin, such as cystine or cysteine.

In particular, we have found that the object is achieved with cosmetic or dermatological preparations which an effective content of
a) at least one antioxidant effective as O- or C-free radical scavenger and
b) at least one organic, boron-containing compound which reduces peroxides or hydroperoxides to the corresponding alcohols without the formation of active free radical consecutive stages.

The preparations according to the invention are suitable in particular for avoiding or reducing skin damage by peroxides or hydroperoxides formed endogenously or exogenously.

The cosmetic or dermatological preparations usually comprise, based on the finished preparations, 0.001 to 30% by weight, preferably 0.01 to 10% by weight and in particular 1 to 5% by weight, of antioxidant (a) and 0.001 to 30% by weight, preferably 0.01 to 10% by weight and in particular 1 to 5% by weight, of at least one peroxide or hydroperoxide decomposer (b).

The peroxide or hydroperoxide decomposers (b) have a significantly greater decomposing (reducing) action than compounds intrinsic to the skin such as cystine or cysteine. Whether certain compounds are suitable for the use according to the invention can be seen in vitro, for example, from the fact that, at room temperature, dissolved in a molar concentration of 0.055 m/l in a polar or nonpolar solvent after storage at 70° C. for 30 minutes, they reduce the peroxide or hydroperoxide concentration by at least 10%, in particular 20%, preferably 50% and in particular 90%. The peroxide or hydroperoxide concentration is usually 0.5 m/l.

The present invention further provides for the use of organic, boron-containing compounds b), which reduces peroxides or hydroperoxides to the corresponding alcohols without the formation of active free radical consecutive stages in cosmetic or dermatological preparations.

The invention further provides for the use of a combination of
a) at least one antioxidant effective as O- or C-free radical scavenger and
b) at least one organic, boron-containing compound which reduces peroxides or hydroperoxides to the corresponding alcohols without the formation of reactive free radical consecutive stages
   in cosmetic or dermatological preparations.

Specifically, suitable boron-containing compounds b) are compounds of the formula (I) 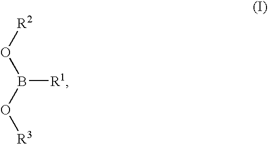
in which the variables, independently of one another, have the following meanings:
R¹, R² and R³:
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted, where the radicals R¹, R² and R³ may be bridged by ring closure.

Examples of compounds of the formula (I) are:

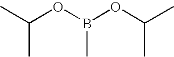

Diisopropoxymethylborane

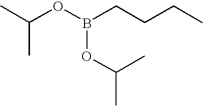

Butyldiisopropoxyborane

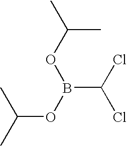

Dichloromethyldiisopropoxyborane

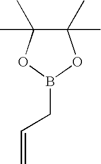

2-Allyl-4,4,5,5-tetramethyl-
1,2,3-dioxaborolane

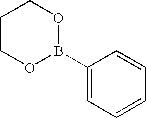

2-Phenyl-1,3,2-dioxyborinane

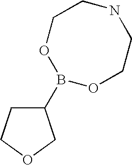

Diethanolamine-(3R)-(+)-tetrahydro-
furanyl boronate

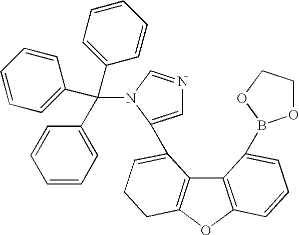

1-(1,3,2-dioxaborolan-2-yl)-9-
(1-trityl-5-imidazolyl)dibenzofuran

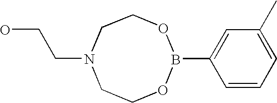

2-(2-M-tolyl-(1,3,6,2)dioxazaboro-
can-6-yl)ethanol

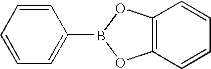

2-Phenyl-1,3,2-benzodioxaborole

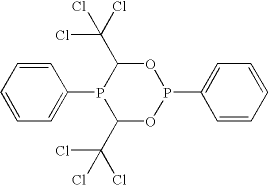

2,5-Diphenyl-4,6-bis-trichloro-
methyl-[1,3,5,2]dioxaphosphaborinane

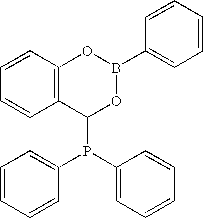

Diphenyl-(2-phenyl-4H-benzo-
[1,3,2]dioxaborinin-4-yl)phosphane

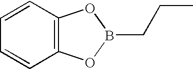

2-Propyl-1,3,2-benzodioxaborole

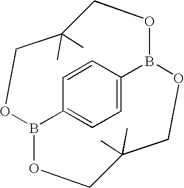

1,4-Benzenediboronic acid bis(neopen-
tyl glycol)cyclic ester

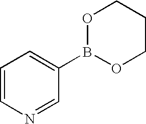

Pyridine-3-boronic acid 1,3-propan-
ediol cyclic ester

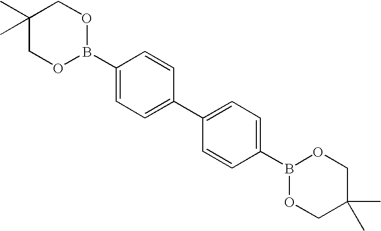

4,4′-Biphenyldiboronic acid bis(neo-
pentyl glycol)cyclic ester

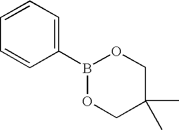

Benzene-boronic acid neopentyl glycol
cyclic ester

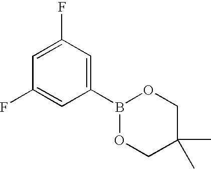

3,5-Difluorobenzeneboronic acid neo-
pentyl glycol cyclic ester

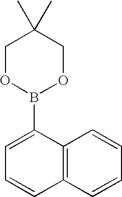

1-Naphthalene boronic acid neopentyl
glycol cyclic ester

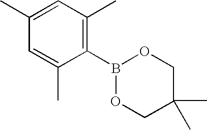

2,4,6-Trimethylbenzene boronic acid
neopentyl glycol cyclic ester

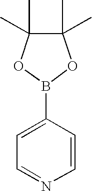

Pyridine-4-boronic acid pinacol cyclic
ester

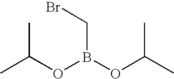

Diisopropyl(bromomethyl) boronate

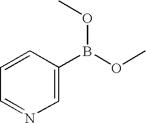

Pyridine-3-boronic acid methyl ester

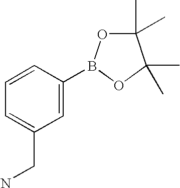

3-Aminomethyiphenylboronic acid,
pinacol ester

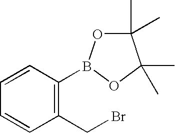

(2-Bromomethylphenyl)boronic acid,
pinacol ester

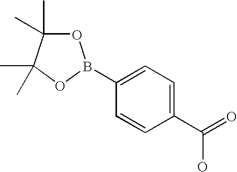

(4-Carboxyphenyl)boronic acid,
pinacol ester

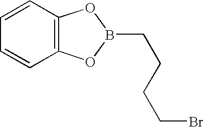

2-(4-Bromobutyl)-1,3,2-benzodioxa-
borole

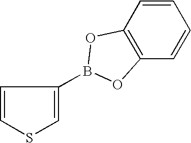

2-(3-Thienyl)-1,3,2-benzodioxybo-
role

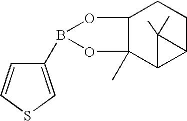

6,6,6B-Trimethyl-2-(3-thienyl)hexa-
hydro-3AH-cyclopropa[E][1,3,2]ben-
zodioxyborole

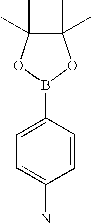

4-(4,4,5,5-Tetramethyl-1,3,2-dioxabo-
rolan-2-yl)aniline

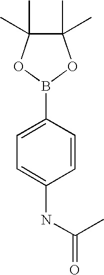

4′-(4,4,5,5-Tetramethyl-1,3,2-dioxa-
borolan-2-yl)acetanilide

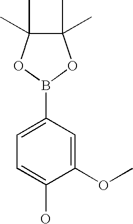

2-Methoxy-4-(4,4,5,5-tetramethyl-
1,3,2-dioxyborolan-2-yl)phenol

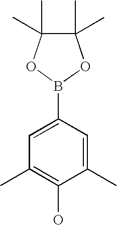

2,6-Dimethyl-4-(4,4,5,5-tetramethyl-
1,3,2-dioxyborolan-2-yl)phenol

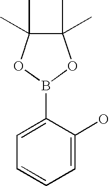

2-(4,4,5,5-Tetramethyl-1,3,2-dioxabo-
rolan-2-yl)phenol

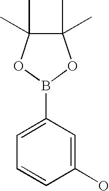

3-(4,4,5,5-Tetramethyl-1,3,2-dioxabo-
rolan-2-yl)phenol

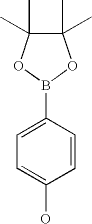

3-(4,4,5,5-Tetramethyl-1,3,2-dioxabo-
rolan-2-yl)phenol

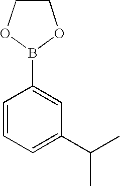

3-Isopropylbenzene acid ethylene gly-
col cyclic ester

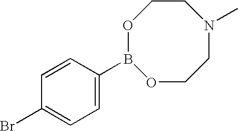

4-Bromobenzeneboronic acid
N-methylbiethanolamine cyclic ester

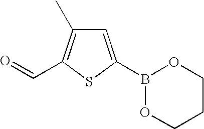

5-Formyl-4-methylthiophene-2-
boronic acid 1,3-propanediol cyclic ester

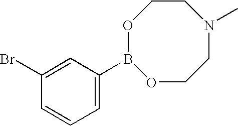

3-Bromobenzeneboronic acid
N-methyldiethanolamine cyclic ester

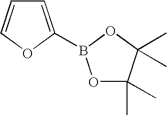

Furan-2-boronic acid pinacol cyclic
ester

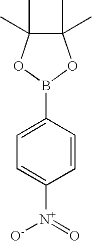

(4-Nitrophenyl)boronic acid, pinacol
ester

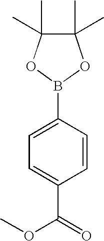

(4-Methoxycarbonylphenyl)boronic
acid, pinacol ester

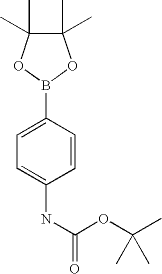

(4-BOC-aminopheny)boronic acid,
pinacol ester

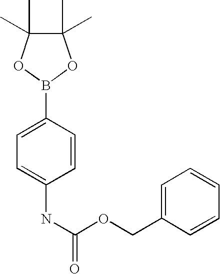

(4-CBZ-Aminophenyl)boronic acid,
pinacol ester

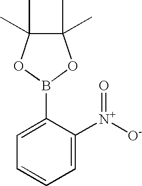

(2-Nitrophenyl)boronic acid, pinacol
ester

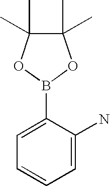

(2-Aminophenyl)boronic acid, pinacol
ester

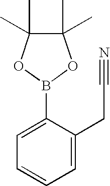

(2-Cyanomethylphenyl)boronic acid,
pinacol ester

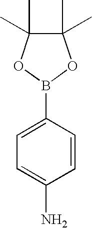

4(4,4,5,5-Tetramethyl-[1,3,2)dioxabo-
rolan-2-yl)phenylamine

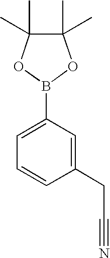

(3-Cyanomethylphenyl)boronic acid,
pinacol ester

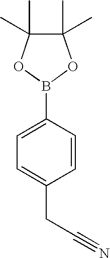

(3-Cyanomethylphenyl)boronic acid,
pinacol ester

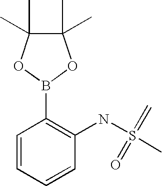

[(2-Methylsulfonyl)aminophenyl]bo-
ronic acid, pinacol ester

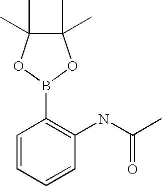

(2-Acetylaminophenyl)boronic acid,
pinacol ester

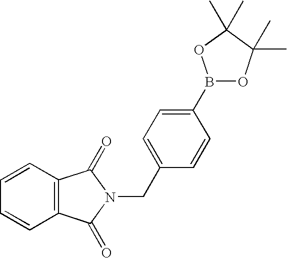

(4-Phthalimidomethylphenyl)boronic
acid, pinacol ester

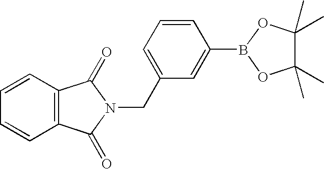

(4-Phthalimidomethylphenyl)boronic
acid, pinacol ester

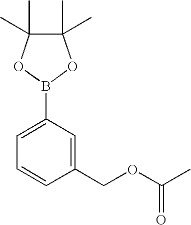

(3-Acetoxymethylphenyl)boronic acid,
pinacol ester

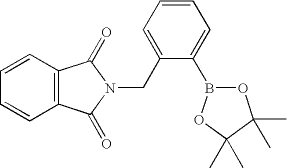

(2-Phthalimidomethylphenyl)boronic
acid, pinacol ester

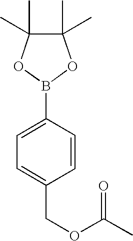

(4-Acetoxymethylphenyl)boronic acid,
pinacol ester

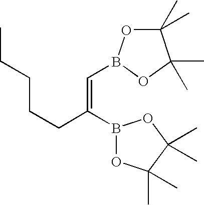

1-[cis-1,2-bis(4,4,5,5-Tetramethyl-
1,3,2-dioxaborolan-2-yl)]heptene

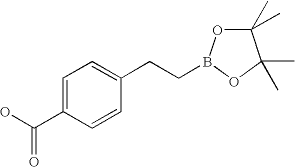

4-(4,4,5,5-Tetramethyl-1,3,2-dioxabo-
rolan-2-yl)ethyl benzoate

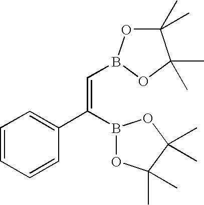

cis-1,2-bis(4,4,5,5-Tetramethyl-
1,3,2-dioxyborolan-2-yl)stilbene

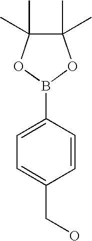

[4-(4,4,5,5-Tetramethyl[1,3,2]dioxa-
borolan-2-yl)-phenyl]methanol

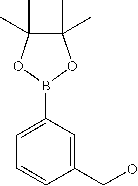

[4-(4,4,5,5-Tetramethyl[1,3,2]dioxa-
borolan-2-yl)-phenyl]methanol

Also suitable as b) are compounds of the formula (II): 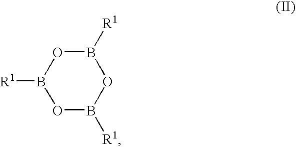
in which R¹ has the meaning given above.

Examples of compounds of the formula (II) are:

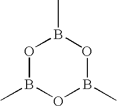

Trimethylboroxin

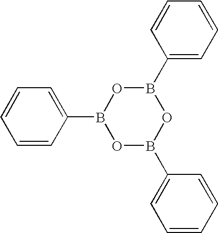

Trimethylboroxin

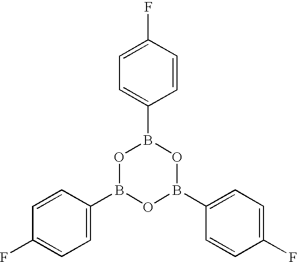

tris(4-Fluorophenyl)boroxin

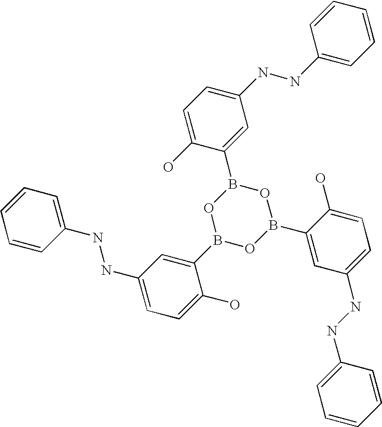

2,4,6-tris(5-(Phenylazo)-2-
hydroxyphenyl)boroxin

Suitable as b) are compounds of the formula (III): 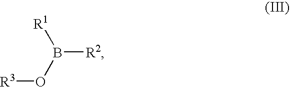
in which R¹, R² and R³ have the meanings given above.

Examples of compounds of the formula (III) are:

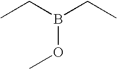

Diethylmethoxyborane

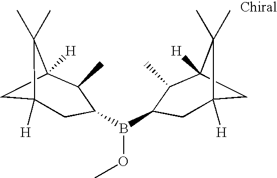

(+)-B-Methoxydiisopino camphylbo-
rane

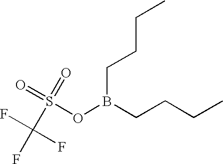

Dibutylborontriflate

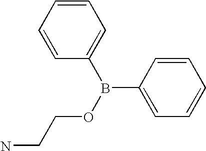

Diphenyl-2-aminoethoxyborane

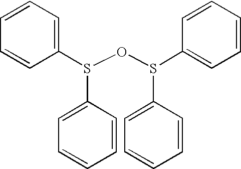

Diphenylborinic anhydride

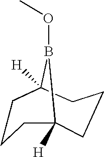

B-Methoxy 9-borabicyclononane

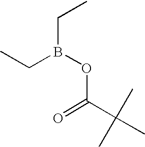

Trimethylacetic acid, anhydride with
diethylborinic acid

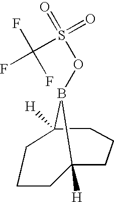

9-BBN-Triflate

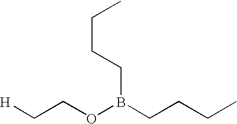

Dibutylboronic acid ethanolamine ester

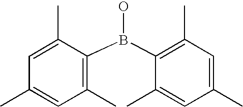

Dimesitylborinic acid

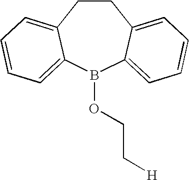

2-(10,11-Dihydro-5H-dibenzo[B,F]-
borepin-5-yloxy)ethylamine

Suitable as b) are compounds of the formula (IV): 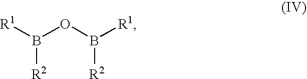
in which R¹ and R² have the meanings given above and R¹ and R² may be bridged by ring closure.

Suitable as b) are compounds of the formula (V): 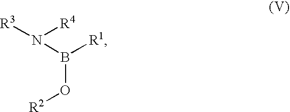
in which R¹, R², R³ have the meanings given above
and R⁴ may have the following meanings
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted, where the radicals R¹, R² and R³ may be bridged by ring closure

Examples of compounds of the formula (V) are:

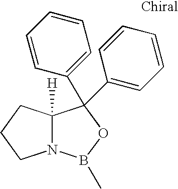

(S)-Methyloxazaborolidine

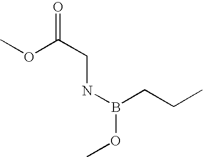

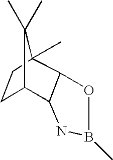

1,4,10,10-Tetramethyl-3-oxa-5-aza-
4-boratricyclo[5.2.1.0(2,6)]decane

Suitable as b) are compounds of the formula (VI):

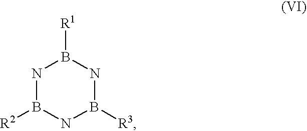

in which R¹, R² and R³ have the meanings given above. Examples of compounds of the formula (VI) are:

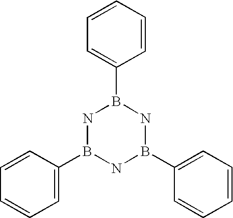

2,4,6-Triphenylborazine

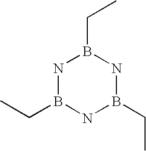

2,4,6-Triethylborazine

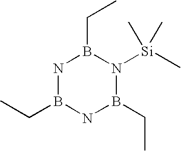

2,4,6-Triethyl-1-(trimethylsilyl)bora-
zine

Suitable as b) are compounds of the formula (VII): 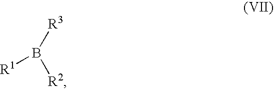
in which R¹, R² and R³ have the meanings given above.

Examples of compounds of the formula VII are:

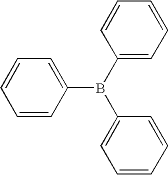

Triphenylborane

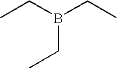

Triphenylborane

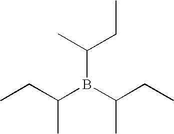

Tri-sec-butylborane

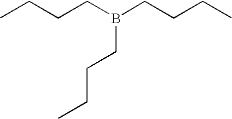

Tributylborane

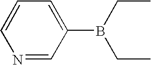

Diethyl(3-pyridyl)borane

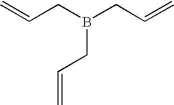

Triallylborane

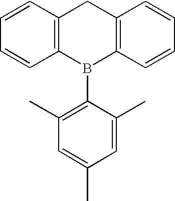

9-(2,4,6-Trimethylphenyl)-9,10-dihy-
dro-9-boraanthracene

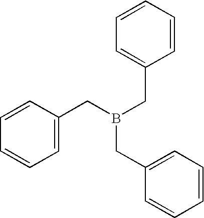

Tribenzylborane

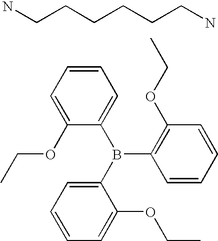

Tris(2-ethoxy-phenyl)borane, com-
pound with hexane-1,6-diamine

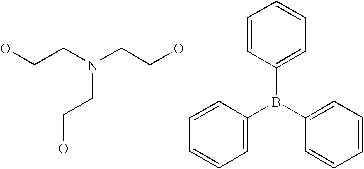

Triphenyl-borane, compound with
2-(bis(2-hydroxyethyl)amino)ethanol

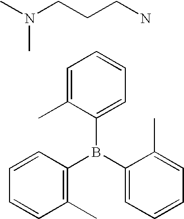

Tri-o-tolylborane, compound with
N(1),N(1)-dimethylpropane-1,3-
diamine

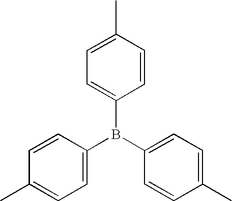

Tri(4-methylphenyl)borane

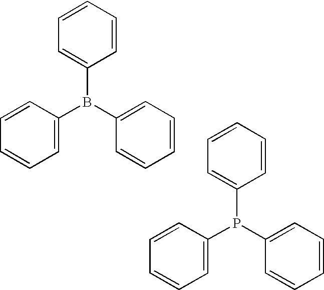

Tetraphenylphosphonium tetraphenyl-
borate

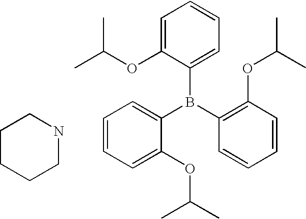

Tris(2-isopropoxyphenyl)borane, com-
pound with piperidine

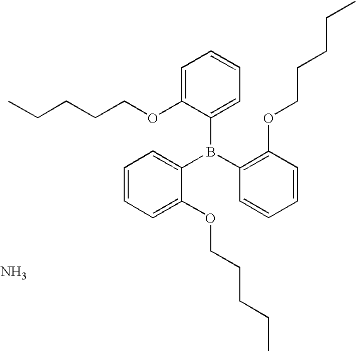

Tris(2-phenyloxyphenyl)borane, com-
pound with ammonia

Suitable as b) are compounds of the formula (VIII): 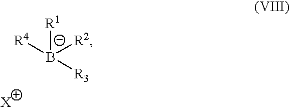
in which the variables, independently of one another, have the following meanings:
R¹, R², R³ and R⁴
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted, where the radicals R¹, R², R³ and R⁴ may be bridged by ring closure
physiologically compatible cations, such as the alkali metal and alkaline earth metal salts or such as optionally substituted ammonium salts.

Examples of compounds of the formula (VIII) are:

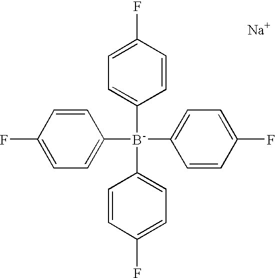

Sodium tetrakis(4-fluorophenyl)borate

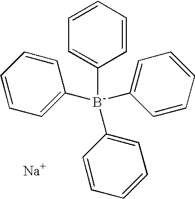

Sodium(tetraphenyl)borate

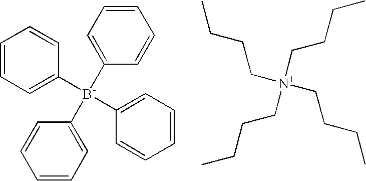

Tetrabutylammonium tetraphenylborate

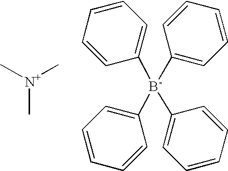

Tetrabutylammonium tetraphenylborate

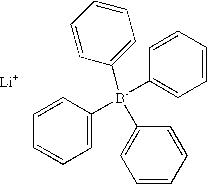

Lithium tetraphenylborate

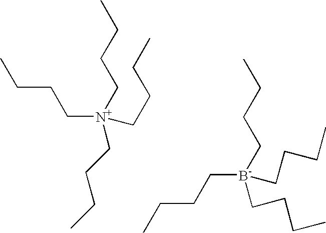

Tetrabutylammonium tetrabutylborate

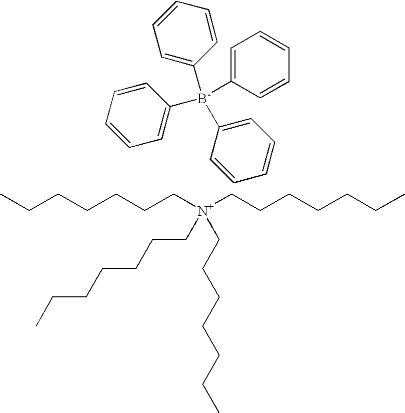

Tetraheptylammonium tetraphenylbo-
rate

Suitable as b) are compounds of the formula (IX): 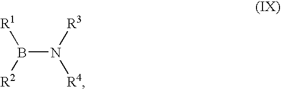
in which the variables, independently of one another, have the following meanings:
R¹, R², R³ and R⁴
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
where the radicals R¹, R², R³ may R⁴ be bridged by ring closure

Suitable alkyl radicals R¹ to R⁴ which may be mentioned are ranched or unbranched C₁-C₂₀-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Particularly preferred alkyl radicals which may be mentioned are methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 2-ethylhexyl.

The alkyl radicals can optionally be substituted by one or more radicals such as halogen (e.g. fluorine, chlorine or bromine), cyano, nitro, amino, hydroxyl or heteroatoms such as sulfur, nitrogen or silicon, the free valences of which may be saturated by hydrogen.

Suitable alkenyl radicals R¹ to R⁴ which may be mentioned are branched or unbranched C₂-C₁₀-alkenyl chains, preferably vinyl, propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 2-hexenyl, 1-heptenyl, 2-heptenyl, 1-octenyl or 2-octenyl.

The radicals R¹ to R⁴ may be bridged by ring closure.

Cycloalkyl radicals which may be mentioned for R¹ to R⁴ are preferably branched or unbranched C₃-C₁₀-cycloalkyl chains, such as cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

Cycloalkenyl radicals which may be mentioned for R¹ to R⁴ are preferably branched or unbranched, C₃-C₁₀-cycloalkenyl chains having one or more double bonds, such as cyclopropenyl, cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecyl.

Particular preference is given to cyclopropyl, cyclopentyl and cyclohexyl. The cycloalkenyl and cycloalkyl radicals can optionally be substituted by one or more, e.g. 1 to 3, radicals, such as halogen (e.g. fluorine, chlorine or bromine), cyano, nitro, amino, C₁-C₄-alkylamino, C₁-C₄-dialkylamino, hydroxyl, C₁-C₄-alkyl, C₁-C₄-alkoxy or other radicals, or contain 1 to 3 heteroatoms, such as sulfur, nitrogen, silicon, the free valences of which may be saturated by hydrogen or C₁-C₄-alkyl, or oxygen in the ring.

Suitable alkoxy radicals are those with 1 to 12 carbon atoms, preferably with 1 to 8 carbon atoms.

Examples which may be mentioned are:

| | |
|---|---|
| methoxy | ethoxy |
| isopropoxy | n-propoxy |
| 1-methylpropoxy | n-butoxy |
| n-pentoxy | 2-methylpropoxy |
| 3-methylbutoxy | 1,1-dimethylpropoxy |
| 2,2-dimethylpropoxy | hexoxy |
| 1-methyl-1-ethylpropoxy | heptoxy |
| octoxy | 2-ethylhexoxy |

Alkoxycarbonyl radicals are, for example, esters which contain the abovementioned alkoxy radicals or radicals of higher alcohols, e.g. with up to 20 carbon atoms such as iso-C₁₅-alcohol.

Suitable mono- or dialkylamino radicals are those which contain alkyl radicals having 1 to 12 carbon atoms, such as, for example, methyl, n-propyl, n-butyl, 2-methylpropyl, 1,1-dimethylpropyl, hexyl, heptyl, 2-ethylhexyl, isopropyl, 1-methylpropyl, n-pentyl, 3-methylbutyl, 2,2-dimethylpropyl, 1-methyl-1-ethylpropyl and octyl.

Aryl is to be understood as meaning aromatic rings or ring systems having 6 to 18 carbon atoms in the ring system, for example phenyl or naphthyl, which may optionally be substituted by one or more radicals, such as halogen, e.g. fluorine, chlorine or bromine, cyano, nitro, amino, C₁-C₄-alkylamino, C₁-C₄-dialkylamino, hydroxyl, C₁-C₄-alkyl, C₁-C4-alkoxy or other radicals. Preference is given to optionally substituted phenyl, methoxyphenyl and naphthyl.

Heteroaryl radicals are advantageously single or fused aromatic ring systems with one or more heteroaromatic 3- to 7-membered rings. Heteroatoms which may be present are one or more nitrogen, sulfur and/or oxygen atoms in the ring or ring system.

Physiologically compatible cations are the cations of the alkali metal and alkaline earth metal salts or of optionally substituted ammonium salts. Examples which may be mentioned are the trialkylammonium salts, such as tri(hydroxyalkyl)ammonium salts or the 2-methylpropan-1-ol-2-ammonium salts. Also suitable are ammonium radicals, in particular alkylammonium radicals.

A choice from the abovementioned compounds is made on the basis of the conditions of skin compatibility or of skin-compatible concentration and the effectiveness of peroxide or hydroperoxide decomposition. For this purpose, the compound under consideration is dissolved in a polar solvent (e.g. acetic acid) or a nonpolar solvent (e.g. toluene) in a molar concentration of 0.055 m/l, and the reaction conversion of a peroxide or hydroperoxide after storage at 70° C. for 30 minutes is measured. In this connection, the concentration of the peroxide or hydroperoxide should be decreased by at least 10%, in particular 20%, preferably 50% and in particular 90%. The peroxide or hydroperoxide concentration is usually 0.5 m/l.

The antioxidants (a) are usually compounds known per se. The antioxidants are advantageously chosen from the group of carotenoids, carotenes (e.g. α-carotene, β-carotene, lycopene) and derivatives thereof, chlorogenic acid and derivatives thereof, lipoic acid and derivatives thereof (e.g. dihydrolipoic acid), and also (metal) chelating agents, EDTA, EGTA and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), butylhydroxytoluene, butylhydroxyanisole, and further antioxidants customarily used in cosmetic preparations.

The amount of abovementioned antioxidants (a) in the finished preparations is, for example, 0.001 to 30% by weight, preferably 0.01 to 10% by weight and in particular 1 to 5% by weight.

The cosmetic and dermatological preparations according to the invention offer effective protection against
oxidative processes,
processes caused by radiation or reactive compounds.

With regard to their other constituents, the novel cosmetic and dermatological formulations can have the customary composition and be used for the treatment, care and cleansing of the skin in cosmetics. The composition depends here on the effectiveness of the inhibitor, the penetration properties of the active substance through the Stratum Corneum and its ability to form a depot in the skin.

Surprisingly, application according to the invention of the active ingredients or ingredient combination makes possible a cosmetically effective treatment, but also prevention of
prematurely aged skin (e.g. wrinkles, age spots, teleangiectases, pigment disorders) and/or prematurely aged skin appendages
radiation-induced skin damage or radiation-induced negative changes in the skin and/or the skin appendages
environmentally induced (ozone, free radicals, singlet oxygen, reactive oxygen or nitrogen compounds, cigarette smoke, toxins) skin damage or environmentally induced negative changes in the skin and/or the skin appendages
light-sensitive, inflammatory, erythematous, allergic or autoimmune reactive changes in the skin and/or the skin appendages (in particular acne, greasy or dry skin, keratoses, rosaceae, dermatoses, atopic eczema, seborrhoic eczema, photodermatoses, polymorphous light dermatosis) deficient, sensitive or hypoactive states of the skin and/or the skin appendages
itching and
dry skin states and horny layer barrier disorders.

For use, the cosmetic and dermatological preparations according to the invention are applied to the skin (and/or the hair) in a sufficient amount in the manner customary for cosmetics.

For example, the active ingredients according to the invention are used in cosmetic compositions for the cleansing of the skin, such as bar soaps, toilet soaps, curd soaps, transparent soaps, luxury soaps, deodorizing soaps, cream soaps, baby soaps, skin protection soaps, abrasive soaps, syndets, liquid soaps, pasty soaps, soft soaps, washing pastes, liquid washing, showering and bath preparations, e.g. washing lotions, shower preparations, shower gels, foam baths, cream foam baths, oil baths, bath extracts, scrub preparations, in-situ products, shaving foams, shaving lotions, shaving creams.

In addition, they are suitable for skin cosmetic preparations, such as W/O or O/W skin and body creams, day and night creams, light protection compositions, aftersun products, hand care products, face creams, multiple emulsions, gelees, microemulsions, liposome preparations, niosome preparations, antiwrinkle creams, face oils, lipogels, sportgels, moisturizing creams, bleaching creams, vitamin creams, skin lotions, care lotions, ampoules, aftershave lotions, preshaves, humectant lotions, tanning lotions, cellulite creams, depigmentation compositions, massage preparations, body powders, face tonics, deodorants, antiperspirants, nose strips, antiacne compositions, repellents and others.

In addition, the active ingredients according to the invention can be used in cosmetic compositions for hair care, such as hair cures, hair lotions, hair rinses, hair emulsions, split-end fluids, neutralizing agents for permanent waves, hot-oil treatment preparations, conditioners, setting lotions, shampoos, hair tints and colorants, hairsprays, blow-waving lotions, blow-waving setting lotions, shine sprays, hair brillantines, hair-styling products, hair tonics, alopecia care compositions and others.

The cosmetic or dermatological preparations can, depending on the field of use, be in the form of a spray (pump spray or aerosol), foam, gel, gel spray, lotion, cream, mousse, ointment, suspensions or powders.

It is also advantageous to administer the active ingredients in encapsulated form, e.g. as cellulose encapsulation, in gelatin, with polyamides, in niosomes, wax matrices, with cyclodextrins or liposomally encapsulated.

The preparations according to the invention generally comprise further auxiliaries as are customarily used in such preparations, e.g. preservatives, bactericides, perfumes, antifoams, dyes, pigments, thickeners, surface-active substances, emulsifiers, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents or silicone derivatives.

In addition to said additives, the preparations according to the invention can comprise further compounds which have an antioxidative, free-radical scavenger, skin moisturizing or moisture-retaining, antierythematous,antiinflammatory or antiallergic action, in order to supplement or enhance their action. In particular, these compounds can be chosen from the group of vitamins, plant extracts, alpha- and beta-hydroxy acids, ceramides, antiinflammatory, antimicrobial or UV-filtering substances, and derivatives thereof and mixtures thereof.

Advantageously, preparations according to the invention can also comprise substances which absorb UV radiation in the UV-B and/or UV-A region.

The lipid phase is advantageously chosen from the group of substances of mineral oils, mineral waxes, branched and/or unbranched hydrocarbons and hydrocarbon waxes, triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures; oils, fats or waxes, esters of saturated and/or unsaturated, branched and/or unbranched C₃-C₃₀-alkane carboxylic acids and saturated and/or unsaturated, branched and/or unbranched C₃-C₃₀-alcohols, from aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil, alkyl benzoates or silicone oils, such as, for example, cyclomethicone, dimethylpolysiloxane, diethylpolysiloxane, octamethylcyclotetrasiloxane and mixtures thereof or dialkyl ethers.

The aqueous phase of the preparations according to the invention optionally advantageously comprises alcohols, diols or polyols of low carbon number, and ethers thereof, preferably ethanol, isopropanol, propylene glycol, glycerol, ethylene glycol monoethyl ether.

Suitable emulsifiers are preferably known W/O and also O/W emulsifiers, such as polyglycerol esters, sorbitan esters or partially esterified glycerides.

Suitable solubility promoters are, in particular, ethoxylated sorbitan esters, ethoxylated lanolin alcohols and ethoxylated castor oil.

Customary native and synthetic thickeners or gel formers in formulations are crosslinked polyacrylic acids and derivatives thereof, polysaccharides, such as xanthane gum or alginates, carboxymethylcellulose or hydroxycarboxymethylcellulose, hydrocolloids such as gum arabic or montmorillonite minerals, such as bentonites or fatty alcohols, polyvinyl alcohol and polyvinylpyrrolidone.

Suitable propellants for aerosols according to the invention are the customary propellants, for example propane, butane, pentane and others.

### EXAMPLE 1

### Measurement of the Peroxide Decomposition

The compounds to be used according to the invention listed in Table 1 and 2 were investigated with regard to their peroxide-decomposing action compared with cystine and cysteine in accordance with the experimental arrangement given below.

Description of the experiment:

The following solutions were prepared:
1. 0.05 molar solution of tert-butyl hydroperoxide in a suitable solvent
2. 0.055 molar solution of the potential hydroperoxide decomposer in a suitable solvent

A suitable solvent may be toluene −d8 or CD₃COOD.

From these, the measurement solutions were prepared by mixing 350 μl of solution 1 and 350 μl of solution 2 in each case; the measurement solution was introduced immediately into an NMR tube and transferred to the NMR instrument. The solutions were always prepared and measurements were always taken at 22° C. Prior to measurement, the solutions were stored in a thermostated bath at 70° C. for 30 minutes. All measurements were carried out using an INOVA 500 500 MHz NMR spectrometer from Varian. For each measurement solution a ¹H-NMR spectrum and a 2D-HSQC (¹H/¹³C) spectrum was recorded. tert-Butyl hydroperoxide and tert-butanol each had CH₃-proton signals which were very close together; assignment of the signals to tBuOOH or tBuOH was made by reference to the 2D-HSQC spectra. The relative proportions of the two components were ascertained by integration of the signal of the corresponding components in the ¹H-spectrum or of the cross peaks in the HSQC spectrum (Lit: W. Wilker et al. Magn. Reson. Chem. 31, 287-292 (1993)).

| | | CD₃-COOD |
|---|---|---|
| | | conversion |
| | | (% t. BuOH) |
| No. | Test substance | 70° C./30 min |
| 1 | Benzeneboronic acid | 100 |
| 2 | Butylboronic acid | 55 |
| 3 | Diisopropoxymethylborane | 17 |
| 4 | Phenyldioxaborinane | 11 |
| 5 | Hydrotris(3-phenylpyrazol-1-yl)borate | 37 |
| 6 | Trimethylboroxin | 17-19 |

### EXAMPLES

### EXAMPLES OF COSMETIC PREPARATIONS

| | | Example |
|---|---|---|
| Formulation type | Field of use | No. |
| O/W emulsion | Soft skin lotion | 1-13 |
| W/O emulsion | Hand protection cream | 14-26 |
| | Sun care lotion | 27-39 |
| Multiple emulsion | W/O/W emulsion | 40-52 |
| Microemulsion | Microemulsion | 53-65 |
| Hydrophilic gel | Liposome gel | 66-78 |
| Lipophilic gel | Blunted oil gel | 79-91 |
| | Oil gel | 92-104 |
| Stick formulation | Sun care lip protection | 105-117 |
| | stick | |
| Aqueous cosmetics | Cooling body splash | 118-130 |
| Decorative cosmetics | Make-up | 131-143 |
| | Liquid make-up | 144-156 |
| Oils | Sun care oil | 157-169 |
| Body cleanser | Facial scrub cleanser | 170-182 |
| Hair aftertreatment | Conditioner | 183-195 |
| agent rinse-off | | |
| Hair aftertreatment | Hair wax | 196-208 |
| agent leave-in | Antidandruff hair tonic | 209-221 |
| Aerosol | Foot deodorant spray | 222-234 |
| | Hairspray | 235-247 |

Formulations 1 to 13—Soft Skin Fluid

| | % w/w |
|---|---|
| Ceteareth-6 and Stearyl Alcohol | 2.50 |
| Ceteareth-25 | 2.50 |
| Hydrogenated Coco-Glycerides | 1.50 |
| PEG-40 Dodecyl Glycol Copolymer | 3.00 |
| Dimethicone | 3.00 |
| Phenethyl Dimethicone | 2.00 |
| Cyclomethicone | 1.00 |
| Cetearyl Octanoate | 5.00 |
| Avocado Oil | 1.00 |
| Sweet Almond Oil | 2.00 |
| Wheat Germ Oil | 0.80 |
| Panthenol USP | 1.00 |
| Phytantriol | 0.20 |
| Tocopheryl Acetate | 0.30 |
| Propylene Glycol | 5.00 |
| Peroxide decomposer according to | 1.00 |
| Example 1 to 6 | |
| Sodium Ascorbyl Phosphate | 2.00 |
| Perfume | q.s. |
| Preservative | q.s. |
| Aqua | ad 100 |

Formulations 14 to 26—Hand Protection Cream

| | % w/w |
|---|---|
| Cetearyl Alcohol | 1.00 |
| Glyceryl Stearate | 1.50 |
| Stearyl Alcohol | 1.50 |
| Cetyl Palmitate | 2.00 |
| Tocopheryl Acetate | 0.50 |
| Dimethicone | 8.00 |
| Ceteareth-6 and Stearyl Alcohol | 3.00 |
| Octyl Methoxycinnamate | 5.00 |
| Propylene glycol | 8.00 |
| Panthenol | 1.00 |
| Evening Primrose Oil | 3.00 |
| PEG-7 Hydrogenated Castor Oil | 6.00 |
| Glyceryl Oleate | 1.00 |
| Phenethyl Dimethicone | 3.00 |
| Beeswax | 1.50 |
| Locust Bean Gum | 0.80 |
| Silk powder | 0.80 |
| Borax | 0.10 |
| Preservative | q.s. |
| Perfume | q.s. |
| Peroxide decomposer according to | 1.20 |
| Example 1 to 6 | |
| Aqua | ad 100 |

Formulations 27 to 39—Sun Care Lotion

| | % w/w |
|---|---|
| PEG-7 Hydrogenated Castor Oil | 6.00 |
| PEG-40 Hydrogenated Castor Oil | 0.50 |
| Isopropyl Palmitate | 7.00 |
| PEG-45/Dodecyl Glycol Copolymer | 2.00 |
| Jojoba Oil | 3.00 |
| Magnesium Stearate | 0.60 |
| Octyl Methoxycinnamate | 8.00 |
| C 12-15 Alkyl Benzoate | 5.00 |
| Titanium Dioxide | 4.00 |
| Propylene Glycol | 5.00 |
| EDTA | 0.20 |
| Preservative | q.s. |
| Sodium Ascorbyl Phosphate | 1.00 |
| Tocopheryl Acetate | 0.50 |
| Peroxide decomposer according to | 0.05 |
| Example 1 to 6 | |
| Perfume | q.s. |
| Aqua | ad 100 |

Formulations 40 to 52—Multiple Emulsion

| | % w/w |
|---|---|
| Mineral Oil | 7.50 |
| Cetearyl Octanoate | 2.50 |
| Aluminum Stearate | 0.25 |
| Magnesium Stearate | 0.25 |
| Microcrystalline Wax H | 0.50 |
| Cetearyl Alcohol | 1.00 |
| Lanolin Alcohol | 1.50 |
| Mineral Alcohol and Lanolin Alcohol | 1.50 |
| PEG-7 Hydrogenated Castor Oil | 0.75 |
| PEG-45/Dodecyl Glycol Copolymer | 2.00 |
| Tocopheryl Acetate | 3.50 |
| Ceteareth-6 and Stearyl Alcohol | 2.00 |
| Ceteareth-25 | 2.00 |
| Trilaureth-4 Phosphate | 1.00 |
| Hydroxyethylcellulose | 0.20 |
| Propylene glycol | 7.50 |
| Magnesium Sulfate | 0.25 |
| Peroxide decomposer according to | 2.00 |
| Example 1 to 6 | |
| Aqua | ad 100 |

Formulations 53 to 65—Microemulsion

| | % w/w |
|---|---|
| Ceteareth-25 | 13.00 |
| PEG-7 Glyceryl Cocoate | 20.00 |
| Octyl Dodecanol | 5.00 |
| Sodium Ascorbyl Phosphate | 0.50 |
| Peroxide decomposer according to | 0.80 |
| Example 1 to 6 | |
| Preservative | q.s. |
| Aqua | ad 100 |

Formulations 66 to 78—Liposome Gel

| | % w/w |
|---|---|
| PEG-40 Hydrogenated Castor Oil | 1.00 |
| Bisabolol rac. | 0.10 |
| Propylene Glycol | 8.00 |
| Panthenol | 0.50 |
| Water and Tocopheryl Acetate and Polysorbate | 3.00 |
| 80 and Caprylic/Capric Triglyceride and Lecithin | |
| Preservative | q.s. |
| Perfume | q.s. |
| Carbomer | 0.50 |
| Peroxide decomposer according to | 0.80 |
| Example 1 to 6 | |
| Triethanolamine | 0.70 |
| Aqua | ad 100 |

Formulations 79 to 91—Blunted Oil Gel

| | % w/w |
|---|---|
| Silica | 5.00 |
| Dimethicone | 10.00 |
| Cetearyl Octanoate | 40.00 |
| Caprylic/Capric Triglyceride | 8.00 |
| Phenethyl Dimethicone | 2.00 |
| Mineral Oil | 26.00 |
| Sweet Almond Oil | 5.00 |
| Tocopheryl Acetate | 1.00 |
| Phytantriol | 0.30 |
| Peroxide decomposer according to | 1.50 |
| Example 1 to 6 | |
| Tocopherol | 0.50 |
| Perfume | 0.70 |

Formulations 92 to 104—Oil Gel

| | % w/w |
|---|---|
| Silica | 5.00 |
| Dimethicone | 10.00 |
| Cetearyl Octanoate | 30.00 |
| Isopropyl myristate | 5.00 |
| Caprylic/Capric Triglyceride | 10.00 |
| Phenethyl Dimethicone | 5.00 |
| Mineral Oil | 25.70 |
| Jojoba Oil | 5.00 |
| Tocopheryl Acetate | 1.00 |
| Phytantriol | 0.30 |
| Peroxide decomposer according to | 1.50 |
| Example 1 to 6 | |
| Tocopherol | 0.50 |
| Perfume | 1.00 |

Formulations 105 to 117—Sun Care Lip Protection Stick

| | % w/w |
|---|---|
| Beeswax | 12.00 |
| Hydrogenated Coco Glycerides | 5.00 |
| Ricinus Oil | 40.00 |
| Isopropyl palmitate | 10.00 |
| Mineral Oil | 7.50 |
| Candellila Wax | 8.00 |
| Phenethyl Dimethicone | 5.00 |
| Tocopheryl Acetate | 1.00 |
| Peroxide decomposer according to | 1.50 |
| Example 1 to 6 | |
| Petrolatum | 5.00 |
| Benzophenone-3 | 5.00 |

Formulations 118 to 130—Cooling Body Splash

| | % w/w |
|---|---|
| PEG-40 Hydrogenated Castor Oil | 2.00 |
| Menthyl Lactate | 0.20 |
| Alcohol | 5.00 |
| PEG-7 Glyceryl Cocoate | 2.00 |
| Witch Hazel | 5.00 |
| Allantoin | 0.10 |
| Bisabolol rac. | 0.20 |
| Propylene glycol | 5.00 |
| Tocopheryl Acetate | 1.00 |
| Sodium Ascorbyl Phosphate | 0.20 |
| Panthenol USP | 0.50 |
| Lactic Acid (80% strength) | 0.20 |
| Peroxide decomposer according to | 2.50 |
| Example 1 to 6 | |
| Perfume | q.s. |
| Aqua | ad 100 |

Formulations 131 to 143—Make-up

| | % w/w |
|---|---|
| Ceteareth-6 and Stearyl Alcohol | 9.00 |
| Dimethicone | 5.00 |
| Cetearyl Octanoate | 8.00 |
| Macadamia Nut Oil | 5.00 |
| Propylene glycol | 5.00 |
| Aqua | 53.00 |
| Sicovit White E 171 | 8.00 |
| Sicomet Brown 70 13E 3717 | 2.00 |
| Tocopheryl Acetate | 0.20 |
| Peroxide decomposer according to | 0.50 |
| Example 1 to 6 | |
| Perfume | q.s. |
| Benzophenone-3 | 4.30 |

Formulations 144 to 156—Fluid make-up

| | % w/w |
|---|---|
| Ceteareth-6 and Stearyl Alcohol | 7.00 |
| Ceteareth-25 | 5.00 |
| Dimethicone | 5.00 |
| Cetearyl Octanoate | 8.00 |
| Macadamia Nut Oil | 5.00 |
| Propylene glycol | 5.00 |
| Aqua | 53.00 |
| Sicovit White E 171 | 8.00 |
| Sicomet Brown 70 13E 3717 | 1.00 |
| Tocopheryl Acetate | 0.20 |
| Peroxide decomposer according to | 0.50 |
| Example 1 to 6 | |
| Perfume | q.s. |
| Benzophenone-3 | 4.30 |

Formulations 157 to 169—Sun Care Oil

| | % w/w |
|---|---|
| Cetearyl Octanoate | 38.00 |
| Caprylic/Capric Triglyceride | 28.20 |
| Evening Primrose Oil | 3.00 |
| Macadamia Nut Oil | 5.00 |
| Isopropyl palmitate | 5.00 |
| Dimethicone | 3.00 |
| Octyl Methoxycinnamate | 8.00 |
| Octocrylene | 5.00 |
| Benzophenone-3 | 2.00 |
| Tocopheryl Acetate | 2.00 |
| Phytantriol | 0.10 |
| Peroxide decomposer according to | 0.50 |
| Example 1 to 6 | |
| Tocopheryl Acetate | 0.20 |
| Perfume | q.s. |

Formulations 170 to 182—Facial Scrub Cleanser

| | % w/w |
|---|---|
| Cocoamidopropyl Betaine | 5.00 |
| Potassium Coco-Hydrolyzed Animal Protein | 7.00 |
| PEG-40 Hydrogenated Castor Oil | 2.00 |
| Polyquaternium-44 | 7.70 |
| Tocopheryl Acetate | 1.00 |
| Bisabolol rac. | 0.20 |
| Panthenol | 1.00 |
| Perfume | 0.50 |
| Hydroxyethyl Cellulose | 2.00 |
| Peroxide decomposer according to | 1.00 |
| Example 1 to 6 | |
| Propylene glycol | 5.00 |
| Jojoba Wax | 3.00 |
| Aqua | ad 100 |

Formulations 183 to 195—Conditioner

| | % w/w |
|---|---|
| Ceteareth-6 and Stearyl Alcohol | 2.00 |
| Ceteareth-25 | 1.00 |
| Cetearyl Octanoate | 6.00 |
| Ceteareth-3 | 2.00 |
| Cetearyl Alcohol | 6.00 |
| Phytantriol | 1.00 |
| Propylene Glycol | 4.00 |
| Polyquaternium-11 | 5.00 |
| Tocopheryl Acetate | 1.00 |
| Panthenol | 1.00 |
| Retinyl Acetate | 0.50 |
| Perfume | q.s. |
| Peroxide decomposer according to | 1.20 |
| Example 1 to 6 | |
| Preservative | q.s. |
| Aqua | ad 100 |

Formulations 196 to 208—Hair Wax

| | % w/w |
|---|---|
| Polyethylene glycol-6 | 30.00 |
| Polyethylene glycol-75 | 45.00 |
| Paraffinum Liquidum | 0.50 |
| PEG-40 Hydrogenated Castor Oil | 1.00 |
| Glycerol | 14.00 |
| Benzophenone-3 | 2.00 |
| Tocopheryl Acetate | 1.00 |
| Phytantriol | 0.10 |
| Peroxide decomposer according to | 1.00 |
| Example 1 to 6 | |
| Perfume | q.s. |
| Aqua | ad 100 |

Formulations 209 to 221—Antidandruff Hair Tonic

| | % w/w |
|---|---|
| Alcohol | 45.00 |
| Aloe Vera (10-fold conc.) | 1.00 |
| Panthenol | 1.00 |
| Tocopheryl Acetate | 0.50 |
| PEG-40 Hydrogenated Castor Oil | 0.50 |
| Allantoin | 0.10 |
| Hydrolyzed Animal Protein | 1.50 |
| 1-(4-Chlorophenoxy)-1-(1H-imidazolyl)-3,3 | 0.30 |
| dimethyl-2-butanone | |
| Perfume | 0.10 |
| Peroxide decomposer according to | 1.00 |
| Example 1 to 6 | |
| Aqua | ad 100 |

Formulations 222 to 234—Foot Deodorant Spray

| | % w/w |
|---|---|
| PEG-40 Hydrogenated Castor Oil | 0.80 |
| Alcohol | 20.00 |
| Farnesol | 0.08 |
| Menthyl Lactate | 0.06 |
| 1,2 Propylene glycol | 3.20 |
| Benzophenone-4 | 1.20 |
| PEG-7 Glyceryl Cocoate | 0.80 |
| Tocopheryl Acetate | 0.05 |
| Peroxide decomposer according to | 0.01 |
| Example 1 to 6 | |
| Perfume | q.s. |
| Aqua | 13.80 |
| Butane | 60.00 |

Formulations 235 to 247—Hairspray

| | % w/w |
|---|---|
| Aminomethyl Propanol | 0.40 |
| Dimethicone Copolyol | 0.03 |
| Alcohol | 43.67 |
| Pentane | 13.20 |
| Acrylates/Acrylamide Copolymer | 3.40 |
| Tocopheryl Acetate | 1.00 |
| Peroxide decomposer according to | 0.01 |
| Example 1 to 6 | |
| Perfume | q.s. |
| Butane | 2.40 |
| Iso-Butane | 35.90 |

1. A cosmetic or dermatological preparation with a content of comprising:
a) at least one antioxidant effective as O- or C-free radical scavenger and
b) at least one organic, boron-containing compound which reduces peroxides or hydroperoxides to the corresponding alcohols without the formation of active free radical consecutive stages, and is chosen from the group consisting of
b1) boron-containing compound of the formula (I) 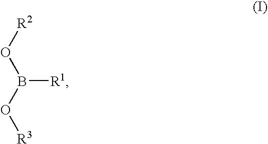
wherein the variables, independently of one another, have the following meanings:
R¹, R² and R³:
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, naphthyl, C₁₈-aryl, aryl, heteroaryl, optionally substituted,
where the radicals R¹, R² and R³ may be bridged by ring closure,
b2) boron-containing compound of the formula (II) 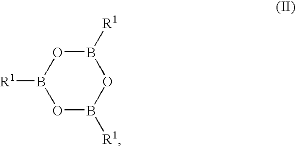
wherein R¹ is
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
b3) boron-containing compound of the formula (III) 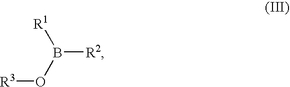
wherein R¹, R² and R³ are
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
where the radicals R¹, R² and R³ may be bridged by ring closure,
b4) boron-containing compound of the formula (IV) 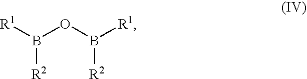
wherein R¹ and R² have the meanings under b3) and R¹ and R² may be bridged by ring closure,
b5) boron-containing compound of the formula (V) 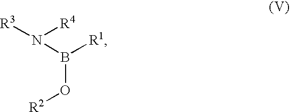
wherein R¹, R² and R³ have the meanings given under b3)
and R⁴ may have the following meanings
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
where the radicals R¹, R², R³ and R⁴ may be bridged by ring closure,
b6) boron-containing compound of the formula (VI) 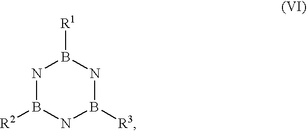
wherein R¹, R² and R³ have the meanings given under b3),
b7) boron-containing compound of the formula (VII) 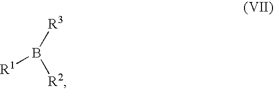
wherein R¹, R² and R³ have the meanings given under b3),
b8) boron-containing compound of the formula (VIII) 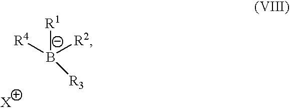
wherein the variables, independently of one another, have the following meanings:
R¹, R², R³ and R⁴
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
where the radicals R¹, R², R³ and R⁴ may be bridged by ring closure,
X^{⊕} physiologically compatible cations, such as the alkali metal and alkaline earth metal salts or such as optionally substituted ammonium salts, and
b9) boron-containing compound of the formula (IX) 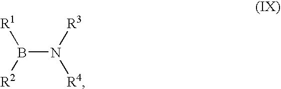
wherein R¹, R², R³ and R⁴ have the meanings given above.

2. The cosmetic or dermatological preparation as claimed in claim 1, comprising, based on the finished preparation, 0.001 to 30% by weight of antioxidant (a) and 0.001 to 30% by weight of at least one boron-containing compound (b).

3. The A cosmetic or dermatological preparation as claimed in claim 1, comprising, as peroxide or hydroperoxide decomposer (b), compounds which, in vitro at room temperature, dissolved in a molar concentration of 0.055 m/l in a polar or nonpolar solvent after storage at 70° C. for 30 minutes, reduce the peroxide or hydroperoxide concentration by at least 10%.

4. The cosmetic or dermatological preparation of claim 1, further comprising one or more auxiliaries.

5. A method of treating and preventing skin damage by peroxides or hydroperoxides formed as a result of endogenous or exogenous factors comprising applying to a skin surface a cosmetic or dermatological preparation, comprising an organic, boron-containing compounds which reduces peroxides or hydroperoxides to the corresponding alcohols without the formation of active free radical consecutive stages.

6. A method for the subsequent elimination and/or alleviation of skin damage by peroxides or hydroperoxides, comprising applying to a skin surface a cosmetic or dermatological preparation, comprising of an organic, boron-containing compounds which reduces peroxides or hydroperoxides to the corresponding alcohols without the formation of active free radical consecutive stages.

7. A method of treating and preventing skin damage by peroxides or hydroperoxides formed as a result of endogenous or exogenous factors, comprising applying to a skin surface a cosmetic or dermatological preparation, comprising a combination of
a) at least one antioxidant effective as O- or C-free radical scavenger and
b) at least one organic, boron-containing compound which reduces peroxides or hydroperoxides to the corresponding alcohols without the formation of reactive free radical consecutive stages, and wherein the boron-containing compound is chosen from the group consisting of
b1) boron-containing compound of the formula (I) 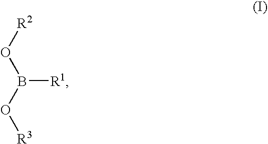
wherein the variables, independently of one another, have the following meanings:
R¹, R² and R³:
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
where the radicals R¹, R² and R³ may be bridged by ring closure,
and boron-containing compounds according to the definitions b2) to b9) as claimed in claim 1.

8. A method for the subsequent elimination and/or alleviation of skin damage by peroxides or hydroperoxides, comprising applying to a skin surface, a cosmetic or dermatological preparation, comprising a combination of
a) at least one antioxidant effective as O- or C-free radical scavenger and
b) at least one organic, boron-containing compound which reduces peroxides or hydroperoxides to the corresponding alcohols without the formation of reactive free radical consecutive stages, where the boron-containing compound is chosen from the group consisting of
b1) boron-containing compound of the formula (I) 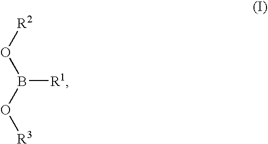
wherein the variables, independently of one another, have the following meanings:
R¹, R² and R³:
hydrogen, C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxycarbonyl, C₁-C₁₂-alkylamino, C₁-C₁₂-dialkylamino, aryl, heteroaryl, optionally substituted,
where the radicals R¹, R² and R³may be bridged by ring closure, and boron-containing compounds according to the definitions b2) to b9) as claimed in claim 1.

9. The preparation of claim 4, wherein 0.001 to 30% by weight of the boron-containing compound is used.

10. A cosmetic or dermatological preparation, comprising an organic, boron-containing compound, which reduces peroxides or hydroperoxides to the corresponding alcohols without the formation of active free radical consecutive stages.

11. The method of claim 5, wherein 0.001 to 30% by weight of the boron-containing compound is used.

12. The method of claim 6, wherein 0.001 to 30% by weight of the boron-containing compound is used.

13. The method of claim 7, wherein 0.001 to 30% by weight of the boron-containing compound is used.

14. The method of claim 8, wherein 0.001 to 30% by weight of the boron-containing compound is used.

15. The preparation of claim 10, wherein 0.001 to 30% by weight of the boron-containing compound is used.
